(19) United States
(12) Patent Application Publication
(10) Pub. No.: US 2014/0329809 A1
(43) Pub. Date: Nov. 6, 2014

(54) NEW LEUKOCYTE INFILTRATE MARKERS FOR ROSACEA AND USES THEREOF

(71) Applicant: GALDERMA RESEARCH & DEVELOPMENT, Biot (FR)

(72) Inventors: Isabelle Carlavan, Grasse (FR); Martin Steinhoff, San Francisco, CA (US)

(73) Assignee: GALDERMA RESEARCH & DEVELOPMENT, Biot (FR)

(21) Appl. No.: 14/354,270

(22) PCT Filed: Oct. 26, 2012
(86) PCT No.: PCT/EP2012/071292

§ 371 Date: Apr. 25, 2014

Related U.S. Application Data
(60) Provisional application No. 61/552,877, filed on Oct. 28, 2011.

(51) Int. Cl. C12Q 1/68; G01N 33/68

(52) U.S. Cl. CPC C12Q 1/6883; G01N 33/6863; G01N 2800/202; G01N 2500/10
USPC 514/229.8; 506/9; 435/6.13; 435/6.12; 435/7.24; 435/6.11; 435/7.92; 436/501; 514/444; 514/320; 514/304; 514/316; 514/278

(57) ABSTRACT

A process for the characterization of rosacea is disclosed. The process can include identifying for the first time new markers in leukocyte recruitment as well as the therapeutic applications targeting rosacea.

The invention is related to a novel characterization process of rosacea by identifying for the first time new markers in the leukocyte recruitment as well as the therapeutic applications targeting in rosacea.

More specifically, the invention proposes the use of CCR1, CCR2, CCR5, CXCR3, CXCR4, CXCR5 and their corresponding ligands involved in leukocyte recruitment as new markers for rosacea, and their use to diagnose rosacea, and/or to screen inhibitors of leukocyte recruitment markers, in particular in inhibiting at least one of these genes and the use of these screened inhibitors in rosacea treatment.

Rosacea is commonly described as a chronic and progressive inflammatory dermatosis related to vascular relaxation. The inflammatory process is characterized by a vascular response to physical and pathogen aggression. In the case of rosacea, this physical response manifests itself by redness of the central part of the face or hot flushes, facial erythema, papules, inflammatory pustules, telangiectasia and sometimes ocular lesions called ocular rosacea. In serious cases, particularly in men, the soft tissue of the nose may swell and produce a bulbous swelling known as rhinophyma. The result of this facial vascular abnormality is a permanent oedema of the dermis, which may be accompanied by an increased colonization by the parasite Demodex folliculorum present on the skin of patients.

Rosacea generally occurs between the ages of 25 and 70, and it is much more common in people with a light complexion. It affects more particularly women, although this condition is generally more serious in men. Rosacea is chronic and persists for years with periods of exacerbation and remission.

According to the National Rosacea Society, rosacea can be classified into four subtypes plus one variant known as granulomatous rosacea. These subtypes are taken up below:

First Subtype—erythematotelangiectatic rosacea:

It is mainly characterized by episodic erythema and persistent central facial erythema. The appearance of telangiectasia is customary but not essential for a diagnosis of this first subtype. Central facial oedema, burning sensations and squamae are also symptoms that have been reported. Conventionally, patients experience erythrosis attacks due to the abrupt dilation of the arterioles of the face, which then takes on a congestive, red appearance. These attacks can in particular be brought on by emotions, meals and changes in temperature.

Second Subtype —papulopustular rosacea:

It is characterized by a persistent central facial erythema with the appearance of central facial papules or pustules. However, the papules and the pustules can also occur in the periorificial regions, i.e. in the perioral, perinasal, or periocular regions. This second subtype resembles common rosacea, except for the fact that the comedones are absent. Burning sensations may also appear. This subtype has often been seen after or in combination with the first subtype. Telangiectasias are often observed after or with the first rosacea subtype. These telangiectasias may be obscured by the erythema, the papules, or the persistent pustules. Some patients also exhibit oedema on the cheeks and the forehead.

Third Subtype—phymatous rosacea

This subtype is characterized by a thickening of the skin and irregular surface nodularities. Rhinophyma most commonly appears, but phymatous rosacea can also appear in other areas such as the chin, the forehead, the cheeks and the ears. Patients suffering from this subtype may also exhibit enlarged and prominent opening of the follicles. This subtype is also often observed after or in combination with subtype 1 or 2, including erythema, telangiectasias, papules and persistent pustules. In the case of rhinophyma, these additional stigmata may be particularly pronounced in the nasal region.

Fourth Subtype—ocular rosacea

The diagnosis of rosacea should be considered when the eyes of a patient show one or more of the following signs and symptoms: bloodshot appearance of the conjunctiva, excessive watering, feeling of a foreign body in the eye, burning, dryness, pruritus, photophobia, blurred vision, conjunctival telangiectasias or eyelid margin telangiectasias, periocular erythema, blepharitis, conjunctivitis, and Meibomius gland dysfunction. These signs or symptoms occur before, during or after the appearance of the cutaneous signs. Ocular rosacea is most commonly diagnosed when other cutaneous symptoms are present. However, the cutaneous signs are not necessary for the diagnosis, and studies suggest that the ocular signs and symptoms can occur, in 20% of cases, before the cutaneous manifestations.

Granulomatous Variant:

There is also a granulomatous variant of rosacea which is characterized by hardened yellow, brown or red papules or nodules, and also monomorphic lesions at the site of the papules. Other signs of rosacea may also be present.

Of course, the pathological manifestations of rosacea vary according to the subtype of the disease. However, it will be noted that patients may have characteristics of several different subtypes at the same time. It will also be noted that the disease does not necessarily progress from one subtype to the other (Wilkin et al., 2002, J. AM. Acad. Dermatol. Vol. 46, pages 584-587).

Many aggressions factors may be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors, vascular factors, or even infection with pathogen Helicobacter pilori).

It has been demonstrated that in Rosacea, neutrophils play an important role not only in the development of inflammatory lesions but also of erythema and telangiectasia (Millikan L. The proposed inflammatory pathophysiology of Rosacea: implications for treatment. Skinmed 2003; 2: 43-47). Moreover, histological observations showed that the inflammatory cell infiltrates around vessels and hair follicles were comprised of predominantly lymphocytes and macrophages but also plasma cells, multinucleated giant cells (Smith J R, Lanier V B, Braziel R M, Falkenhagen K M, White C, Rosenbaum J T. Expression of vascular endothelial growth factor and its receptors in rosacea. Br J Ophthalmol. 2007 February; 91(2):226-9.). A dermal infiltrates with a predominance of CD4+ T helper (TH) cells over CD8+ T cells is observed in Rosacea lesions with an association of Demodex folliculorum (DF), supporting the hypothesis that cell-mediated immune responses play an important role in the pathogenesis of the disease (Rufli T, Büchner S A. T-cell subsets in acne rosacea lesions and the possible role of Demodex folliculorum. Dermatologica. 1984; 169(1):1-5).

Thus, inflammatory events are a key cause of rosacea.

Chemokines are capable of selectively inducing chemotaxis of leukocytes such as neutrophils, monocytes, macrophages, eosinophils, basophils, mast cells, and lymphocytes, such as T cells and B cells. Chemokines have been classified into 4 supergene families based on the location of cysteine residues. The 4 groups are CXC, CC, C, and CX3C chemokines (Rossi D, Zlotnik A. The biology of chemokines and their receptors. Annu Rev Immunol 2000; 18: 217-42, Bacon K, Baggiolini M, Broxmeyer H, Horuk R, Lindley I, Mantovani A, et al. Chemokine/chemokine receptor nomenclature. J Interferon Cytokine Res 2002; 22: 1067-8).

Most CXC-chemokines attract neutrophil leukocytes such as CXCL8 or interleukin-8 (IL-8) and CXCL1 (GRO alpha). Some CXC-chemokines such as CXCL9 (Mig or monokine induced by gamma interferon) and CXCL10 (IP-10 or interferon-gamma inducible 10 kDa protein) are particularly active in inducing chemotaxis of activated peripheral blood lymphocytes (Esche C, Stellato C, Beck LA. Chemokines: key players in innate and adaptive immunity. J Invest Dermatol. 2005 October; 125(4):615-28; Moser B, Willimann K. Chemokines: role in inflammation and immune surveillance. Ann Rheum Dis. 2004 November; 63 Suppl 2:ii84-ii89.).

CC-chemokines are generally less selective and can attract a variety of leukocyte cell types, including monocytes, eosinophils, basophils, T lymphocytes and natural killer cells.

Chemokines interact with receptors found predominantly on the surface of leukocytes. These receptors are G protein-coupled receptors containing 7 transmembrane domains. Chemokine receptors, such as CCR1, CCR2, CCR2A, CCR2B, CCR3, CCR4, CCR5, CCR6, CCR7, CCR8, CCR9, CXCR1, CXCR2, CXCR3, CXCR4, CXCR5, CX3CR1, and XCR1 have been implicated as being important mediators of inflammatory and immunoregulatory disorders and diseases, including asthma and allergic diseases, as well as autoimmune pathologies such as rheumatoid arthritis and atherosclerosis.

By regulating the migration and activation of leukocytes from the peripheral blood to inflammatory sites, the chemokines, mentioned below, play a critical role in the maintenance of host defense as well as in the development of the immune response in lesional skin of rosacea patients.

CXC chemokine receptor 3 (CXCR3), previously referred G protein-coupled receptor 9 (GPR9) or CD183, is a Gai protein-coupled receptor. CXCR3 is expressed primarily on T lymphocytes, preferentially type I T helper cells. CXCR3 binds selectivity three chemokines, termed CXCL10 or IP10 (interferon-g-inducible 10 kDa protein), CXCL9 or Mig (monokine induced by interferon-g) and CXCL11 or I-TAC (interferon-inducible T cell a-chemoattractant) induced primarily by IFN-γ and produced by macrophages as well as other cell types in inflamed tissue. Binding of chemokines to this protein induces cellular responses that are involved in leukocyte recruitment, most notably integrin activation, cytoskeletal changes and chemotactic migration.

CXC chemokine receptor 4 (CXCR4), also known as fusin or CD184, is a Gi protein-coupled receptor. CXCR4 is the specific receptor for stromal-derived-factor-1 (SDF-1 also called CXCL12), a molecule endowed with potent chemotactic activity for lymphocytes and monocytes. CXCR4 mRNA is constitutively expressed in almost all types of leukocytes.
C-X-C chemokine receptor type 5 (CXCR5 or CD185) also known as Burkitt lymphoma receptor 1 (BLR1) is a G protein-coupled seven transmembrane receptor for chemokine CXCL13 (also known as BLC). This receptor is mainly expressed on mature B-lymphocytes and Burkitt's lymphoma cells. CXCLI3 is known to attract naive B-cells and certain activated and memory T-cells.
C-X-C chemokine receptor type 6 (CXCR6 or CD186) is a seven transmembrane G protein coupled receptor, expressed on Th1 cells and NKT cells but not by Th2 cells, establishing CXCR6 as a differential marker of polarized type 1 T helper cells. The ligand of this receptor, CXCL16, is produced by several cells, including dendritic cells, macrophages, B-cells, T-cells, smooth muscle cells, endothelial cells, bone marrow stromal cells, neuronal cells, epithelial cells and fibroblasts.
C-C chemokine receptor type 1 (CCR1 or CD191) is a G protein-coupled receptor, expressed on monocytes, T cells, dendritic cells, and, in some cases, on neutrophils. The binding of at least three chemokines, MIP-1 alpha/CCL3, MCP3/CCL7 and RANTES/CCL5 to CCR1 is responsible for the trafficking of monocytes, macrophages and TH1 cells to inflamed tissues.
C-C chemokine receptor type 2 (CCR2 or CD192) is found on the surface of monocytes, macrophages, B cells, activated T cells, dendritic cells, endothelial cells and tumor cells. It is a receptor for a number of chemokine ligands, including MCP-1 (CCL2), MCP-2 (CCL8), MCP-3 (CCL7) and MCP-4 (CCL13). CCR2 mediates migration of monocytes, antigen-presenting cells or dendritic cells and lymphocytes to various tissues under inflammatory conditions.
C-C chemokine receptor type 5 (CCR5 or CD195) is a G protein-coupled receptor, member of the beta chemokine receptors family of integral membrane proteins. The natural chemokine ligands that bind to this receptor are RANTES (CCL5) and macrophage inflammatory protein (MIP) 1α (CCL3) and 1β (CCL4). CCR5 is predominantly expressed on T cells (preferentially Th type I cells), macrophages, dendritic cells and microglia.

The Uniprot references are respectively : CCR1: P32246; CCR2: P41597; CCR5: P51681; CXCR3: P49682; CXCR4: P61073; CXCR5: P32302; CXCR6: 000574; CXCL9: Q07325; CXCL10: P02778; CXCL11: 014625; CXCL12: P48061; CXCL13: Q53X90; CXCL16: Q9H2A7; CCL2: P13500; CCL3: P10147; CCL4: P13236; CCL5: P13501; CCL7: P80098; CCL13: Q99616.

In this context, for the first time, the applicant proposes with experimental evidences to target rosacea markers, CCR1, CCR2, CCR5, CXCR3, CXCR4, CXCR5, CXCR6 and their corresponding ligands CXCL9, CXCL10, CXCL11, CXCL12, CXCL13, CXCL16, CCL2, CCL3, CCL4, CCL5, CCL7, CCL13, which are responsible for the leukocyte recruitment for treating and diagnosing rosacea.

Thus, the invention is relating to the use of at least one of the DNA or the mRNA encoding CCR1, CCR2, CCR5, CXCR3, CXCR4, CXCR5, CXCR6 and also the corresponding proteins, as markers for rosacea as well as the use of the DNA or the mRNA encoding at least one of the chemokines, ligands of chemokine receptors mentioned above, chosen from CXCL9, CXCL10, CXCL11, CXCL12, CXCL13, CXCL16, CCL2, CCL3, CCL4, CCL5, CCL7, CCL13 and also the corresponding proteins, as markers for rosacea.

The invention also provides a method for the diagnosis of rosacea, comprising the following steps:
a) detecting the level of expression of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13 in a sample taken from an individual,
b) detecting the level of expression of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13 in a sample taken from a healthy individual,
c) comparing the difference in level of expression of at least one marker and for which the level of expression is significantly higher than the level of expression in the healthy individual;
d) the overexpression of at least one of the markers of step c) being an indicator of rosacea, thus diagnosing rosacea.

The invention provides also a method for the diagnosis of rosacea that can also comprise the following steps:
a) detecting the level of expression of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13 in a sample taken from an individual,
b) detecting the level of expression of at least one of the proposed markers selected from CCR1, CCR2, CCR5, CXCR3, CXCR4, CXCR5 and CXCR6 and/or at least one of the markers chosen from CXCL9, CXCL10, CXCL11, CXCL12, CXCL13, CXCL16, CCL2, CCL3, CCL4, CCL5, CCL7, and CCL13 in a sample taken from a normal individual,
c) comparing the difference in level of expression of at least one marker and for which the level of expression is significantly higher than the level of expression in the healthy individual;
d) the overexpression of at least one of the markers of step c) being an indicator of rosacea, thus diagnosing rosacea.

The invention provides a method for monitoring the progression or variation of rosacea, comprising the following steps:
a) taking a biological sample from the individual,
b) analysing the level of expression of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13 in a sample taken and in which a variation in the expression of at least one of the markers is an indicator of the progression of rosacea.

The invention provides also a method for monitoring the efficacy of a treatment intended for treating rosacea, comprising the following steps:
a) administering the desired treatment to the individual identified as having one or more of the symptoms of rosacea,
b) taking a biological sample from the individual,
c) analysing the level of expression of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13, in which a variation in the expression of at least one of the markers is an indicator of efficacy in the treatment of rosacea.

The invention provides also an in vitro screening method of leukocyte recruitment inhibitors for treating rosacea, comprising determining the capacity of said candidate to inhibit or down regulate expression and/or the biological function of one of the proposed markers.

More specifically, the invention relates to an in vitro screening method of leukocyte recruitment inhibitors for the identification of drug candidates, comprising the following steps:
a) Collecting at least two biological samples: one mimics the rosacea lesion, and one mimics the healthy condition;
b) Contacting at least one sample or a mixture of samples with one or more drug candidates to be tested;
c) Detecting the expression or biological function of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13 in the biological samples or mixture obtained in b);
d) Selecting drug candidates which are capable of inhibiting the expression or biological function of at least one of the proposed markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7 and CCL13 measured in said samples or mixtures obtained in b) and comparing the levels with a sample not mixed with the drug candidate (s).

In another embodiment, the invention provides an in vitro screening method of leukocyte recruitment inhibitors for drug candidate identification, comprising the following steps:
a) Collecting at least two biological samples: one mimics the rosacea lesion, and one mimics the healthy condition;
b) Contacting at least one sample or a mixture of samples with one or more drug candidates to be tested;
c) Detecting the expression or biological function of at least one of the proposed markers in the biological samples or mixture obtained in step b);
d) Selecting drug candidates which are capable of inhibiting the expression or biological function of at least one marker chosen from the proposed markers measured in said samples or mixture obtained in step b) and comparing the levels or biological function with a sample not mixed with the drug candidate.

The invention relates also to the use of inhibitors identified by screening methods as defined above for the preparation of a composition for treating rosacea and/or rosacea associated disorders. More specifically, the invention encompasses the use of inhibitors of the proposed markers identified by screening methods for the preparation of a composition for treating rosacea or rosacea associated disorders such as:

CCR1 antagonists: 2-thiophen-2-yl-5-[5-[5-(5-thiophen-2-ylthiophen-2-yl)thiophen-2-yl]thiophen-2-yl]thiophene; 1,4-cis-1-(1-Cycloocten-1-ylmethyl)-4-[[(2,7-dichloro-9H-xanthen-9-yl)carbonyl]amino]-1-ethylpiperidinium iodide, (7R,7aS)-2-Chloro-4-(7-hydroxy-1,3-dioxotetrahydropyrrolo[1,2-c]imidazol-2-yl)-3-methylbenzonitrile, [5-chloro-2-[2-[(2R)-4-[(4-fluorophenyl)methyl]-2-methylpiperazin-1-yl]-2-oxoethoxy]phenyl]urea hydrochloride, compounds disclosed in patents WO2010/036632, WO2009/134666 and WO2009/137338, WO98/56771, US/6812230, US 2008/0139602, WO2003/105853, WO2009/082526, WO1998/038167, WO2008/011392, WO2011/056440, WO2011/049917, WO/2006/133802, WO2008/103126 and WO2009/011653;
CCR2 antagonists: (5E)-8-[4-(2-butoxyethoxy)phenyl]-1-(2-methylpropyl)-N-[4-[(3-propylimidazol-4-yl) methylsulfinyl]phenyl]-3,4-dihydro-2H-1-benzazocine-5-carboxamide; methanesulfonic acid, 6-Methyl-1′-[2-(5-methyl-2-phenyl-4-oxazolyl)ethyl]-spiro[4 H-3,1-benzoxazine-4,4′-piperidin]-2(1H)-one, 2-[(Isopropylaminocarbonyl)amino]-N-[2-[[cis-2-[[4-(methylthio)benzoyl]amino]cyclohexyl]amino]-2-oxoethyl]-5-(trifluoromethyl)benzamide, 1′-[2-[4-(Trifluoromethyl)phenyl]ethyll]-spiro[4H-3,1-benzoxazine-4,4′-piperidin]-2(1H)-one hydrochloride, compounds disclosed in patents WO2009/076404, WO2006/012135, WO2004/069810, WO2006/036527, WO2008/109238, WO2008/145681, WO2007/130712, WO2007/106797, WO2005/118574, WO2006/015986, WO2011/073155, WO2011/073154, WO2011/042399, WO2010/070032, WO2007/053495, WO2007/053498, WO2007/053499, WO2010/068663, WO2010/121011, WO2010/121046, WO2009/003861, WO2010/121036, WO2006/076644, WO2008/008375, WO2008/010934, WO2010/074409;
CCR5 antagonists: 4,4-difluoro-N-[(1S)-3-[(1R,5S)-3-(3-methyl-5-propan-2-yl-1,2,4-triazol-4-yl)-8-azabicyclo[3.2.1]octan-8-yl]-1-phenylpropyl]cyclohexane-1-carboxamide, (5E)-8-[4-(2-butoxyethoxy)phenyl]-1-(2-methylpropyl)-N-[4-[(S)-(3-propylimidazol-4-yl)methylsulfinyl]phenyl]-3,4-dihydro-2H-1-benzazocine-5-carboxamide, (4-nitrophenyl)methyl N-[1-[[(3S,4 R)-1-(cyclopentanecarbonyl)-4-hydroxy-4-phenylpyrrolidin-3-yl]methyl]piperidin-4-yl]-N-prop-2-enylcarbamate, 1-acetyl-N-[3-[4-[(4-carbamoylphenyl)methyl]piperidin-1-yl]propyl]-N-(3-chloro-4-methylphenyl)piperidine-4-carboxamide, (4,6-dimethylpyrimidin-5-yl)-[4-[(3S)-4-[(1R)-2-methoxy-1-[4-(trifluoromethyl)phenyl]ethyl]-3-methylpiperazin-1-yl]-4-methylpiperidin-1-yl]methanone, 40[4-[[(9R)-11-butyl-9-[(R)-cyclohexyl(hydroxy)methyl]-7,10-dioxo-3,8,11-triazaspiro[5.5]undecan-3-yl]methyl]phenoxy]benzoic acid;
CXCR3 antagonists: N-[(1R)-1-[3-(4-ethoxyphenyl)-4-oxopyrido[2,3-d]pyrimidin-2-yl]ethyl]-N-(pyridin-3-ylmethyl)-2-[4-(trifluoromethoxy)phenyl]acetamide, compounds disclosed in patents: WO2007/062175, WO2002/083143, WO2009/094168, WO2002/085861, WO2004/075863, WO/2006/088836, WO2008/079279, WO2011/084985, WO2007/090826, WO2007/090836, WO2008/008453, WO2007/109238, WO2007/047202, WO2006/088921, WO2006/088840, WO2006/088920, WO2006/088919, WO2006/091428, WO2007/002742, WO2007/064553;
CXCR4 antagonists: 1-[[4-(1,4,8,11-tetrazacyclotetradec-1-ylmethyl)phenyl]methyl]-1,4,8,11-tetrazacyclotetradecane, N-(pyridin-2-ylmethyl)-1-[3-(1,4,8,11-tetrazacyclotetradec-1-ylmethyl)phenyl]methanamine, compounds disclosed in patents: WO2004/087068, WO2006/074428, WO2008/008852, WO2006/126188, WO2006/074426, WO2008/150689, WO2010/025416, WO2009/004054, WO2007/074871, WO2008/008854, WO2006/074426;
CXCR5 antagonists: compounds disclosed in patents: WO/2010/053547, WO/2008/151211.

For the purpose of the present invention, the term “marker” or “biological marker” denotes a biological marker associated with the presence or with the absence of a particular pathological state. The biological markers are in particular proteins, mRNAs or DNAs.

The term “level of expression” or “expression” means the level of mRNAs or proteins encoded by the gene marker.

The expression level analysis or detection can be performed by any suitable method, known to those skilled in the art, such as western blotting, IHC, mass spectrometry (Maldi-TOF and LC/MS analyses), radioimmunoassay (RIA), Elisa or any other method known to those skilled in the art or else by assaying the mRNA according to the methods customarily known to those skilled in the art. The techniques based on the hybridization of mRNA with specific nucleotide probes are the most customary (Northern blotting, RT-PCR (Reverse Transcriptase Polymerase Chain Reaction), quantitative RT-PCR (qRT-PCR), RNase protection).

Progression of rosacea may be from a predominantly vascular to a more inflammatory dominated state, it may also mean progression towards specific rosacea subtypes as described above. Progression might also occur in the other direction, from a more severe to a less severe form of rosacea.

Thus, the invention relates also to a method for the prognosis of the progression or variation of rosacea.

The expression “overexpression of one of the factors or markers” is intended to mean a level of expression increased by at least 50%, and preferably by at least 100%, and even more preferably by at least 200%, or expressed differently with equivalent significance, by at least a factor of 2, or at least twice as high as the level in a normal individual; which demonstrates overall an overexpression of the chemokines, the cytokines and the receptors mentioned above, thus representing markers characteristic of rosacea.

For the screening, biological samples are transfected cells containing reporter gene operably under the control of a promoter (totally or partially) controlling the expression of an above mentioned gene. Therefore step c) above consists to measure the expression of the reporter gene.

The reporter gene may encode an enzyme that with its corresponding substrate, provides coloured product(s) such as CAT (chloramphenicol acetyltransferase), GAL (beta galactosidase), or GUS (beta glucuronidase). It might be either luciferase or GFP (Green Fluorescent Protein) gene.

Reporter gene protein dosage or its activity is typically assessed by colouring, fluorometric or chemoluminescence methods.

According to a second embodiment of the invention, biological samples are cells expressing the gene of interest and the step c) above consists to measure the activity of the gene product.

Any kind of cell is suitable for the invention. Cells may endogenously express the said gene like lymphocytes. Organs may be suitable for the instant invention, from animal or human origin like lymph nodes.

Transformed cells by heterologous nucleic acid encoding the gene expression product of interest might be suitable. Preferably the said nucleic acid is from animal (preferred mammal) or human origin. A large variety of host cells is suitable for the invention and in particular Cos-7, CHO, BHK, 3T3, HEK293 cells. Cells are transiently or permanently transfected by a nucleic acid of interest with a well known by skilled in the art method and for instance calcium phosphate precipitation, DEAE-dextran, liposome, virus, electroporation or microinjection. The gene expression of step c) is determined with the same techniques quoted above for diagnostic.

The compounds to be tested are any kind of compounds, from natural or synthetic source. As synthetic compounds they might be chemically synthesized or from chemical compound data bank, with a defined structure or non characterized or present in a mixture of compounds.

Several technical assays are available for assessing compounds activity modulating above mentioned biomarkers/gene expression products.

In other embodiment, the invention is related to the use of identified inhibitors with the described screening methods for the preparation of a composition for treating rosacea or rosacea associated disorders.

In the context of the invention, the biological sample corresponds to any type of sample taken from an individual, and can be a tissue sample or a fluid sample, such as blood, lymph or interstitial fluid.

According to one particular and preferred embodiment, the sample is a biopsy of varying size (preferably from 1 to 6 mm in diameter), or a skin sample taken by means of tape stripping, such as with D-Squames, according to the method described in Wong R et al., “Analysis of RNA recovery and gene expression in the epidermis using non-invasive tape stripping”; J Dermatol Sci. 2006 November; 44(2):81-92; or in Benson N R, et al., “An analysis of select pathogenic messages in lesional and non-lesional psoriatic skin using non-invasive tape harvesting”. J Invest Dermatol. 2006 October; 126(10): 2234-41; or else in Wong R et al., “Use of RT-PCR and DNA microarrays to characterize RNA recovered by non-invasive tape harvesting of normal and inflamed skin”. J Invest Dermatol. 2004 July; 123(1):159-67. According to the principle of tape stripping, the product used comprises a flexible translucent polymer support and an adhesive. The product is applied repeatedly to the skin of the patient, preferably until loss of adhesion. The sample obtained relates only to the content of the outermost layers of the epidermis. A method for analysing a protein content obtained in particular according to this sampling method is described in Patent Application WO2009/068825 (Galderma R&D) in order to monitor markers specific for a pathological skin condition and to orient the diagnosis. Since this method is rapid, non-invasive and relatively inexpensive for detecting the presence of, the absence of or the variation in certain proteomic markers, it is particularly preferred. This method is in particular characterized by mass spectrometry detection, ELISA or any other method known to the expert skilled in the art of protein quantification. Quantification is performed in the skin sample obtained on the flexible and adhesive support in order to detect at least one protein of which the presence, the absence or the variation in amount or in concentration compared with a standard value is associated with the presence, with the progression or with the absence of a particular pathological skin condition.

Another embodiment of the present invention is an in vitro screening method of leukocyte recruitment candidate inhibitors, comprising determining the capacity of said candidate to inhibit and/or down regulate the expression or the biological activity or the biological function, including the transactivation properties, of at least one of the proposed markers of the invention.

According to a further embodiment of the invention, biological samples are cells expressing the gene of interest and the step c) above consists to measure the activity of the gene product.

In another embodiment, the invention is related to the use of identified inhibitors/antagonists/inverse agonists with the described screening methods for the preparation of a composition for treating rosacea and/or rosacea associated disorders.

In other aspect, inhibitors might be either a polypeptide, a DNA or RNA antisens, a si-RNA or a PNA (Peptide nucleic acid), i-e with a polypeptidic chain substituted by purine and pyrimidine bases and having a DNA -like structure for hybridization to this latter).

The modulator might be an antibody and preferably a monoclonal antibody. Advantageously, the monoclonal antibody is administered to a patient in a sufficient quantity so as the measure a plasmatic concentration from about 1 μg/ml to about 100 μg/ml, preferred from about 1 μg/ml to about 5 μg/ml.

The invention is intended for treating rosacea. By rosacea it is understood, all rosacea subtypes as well as rosacea associated disorders.

The example which follows illustrates the invention without limiting the scope thereof.

### EXAMPLE

Analysis of the expression of CCR1, CCR2, CCR5, CXCR3, CXCR4, CXCR5, CXCR6 CXCL9, CXCL10, CXCL11, CXCL12, CXCL13, CXCL16, CCL2, CCL3, CCL4, CCL5, CCL7 and CCL13 in the lesional skin of patients suffering from rosacea compared with non lesional skin of these patients.

### Patient Selection and Tissue Biopsie:

Skin biopsies of rosacea patients with rosacea subtype I (n=11), II (n=11) and III (n=6) were performed, in accordance with good clinical practice. (The clinical description of rosacea subtypes was carried out according to the classification of Wilkin et al., 2002, J. Am. Acad. Dermatol. Vol 46, pages 584-587.)

To evaluate a change in the expression level of the genes, the expression levels in lesional skin are compared with the expression levels in non-lesional skin of the same subjects (n=12).

mRNA Extraction, Labelling and Hybridization to Probe Arrays:

The mRNA was isolated from skin using the RNeasy extraction kit (Quigen Inc., Valencia, Calif.) and quality was evaluated using a 2100 Bioanalyser of Agilent. The mRNA expression was evaluated by a Gene Chip IVT labelling kit after the generation of double-stranded cDNA (i.e in vitro transcription process) using T7-oligo primer and the one cycle cDNA synthesis kit of Affymetrix. RNA was ethanol precipitated to concentrate the sample and then quantified using a spectrophotometer. Approximately 200 ng of total RNA of good quality [RNA indication number (RIN)≧7] from each sample was used to generate double-stranded cDNA using a T7-oligo (dt) primer (one cycle cDNA synthesis kit, Affymetrix). Biotinylated cRNA, produced through in vitro transcription (Gene Chip IVT labelling kit, Affymetrix) was fragmented and hybridised to an Affymetrix human U133A 2.0 plus microarray. The arrays were processed on a Gene Chip Fluidics Station 450 and scanned on an Affymetrix Gene Chip Scanner (Santa Clara, Calif.).

Statistical Analysis of mRNA Expression:

The expression data from Affymetrix Gene Chips are normalized with RMA (Robust Multi-array Analysis) method. The raw intensity values are background corrected, log2 transformed and then quantile normalized. Next a linear model is fit to the normalized data to obtain an expression measure for each probe set on each array. To identify genes that were significantly modulated in the different Rosacea subtype samples, one-way ANOVA with Benjamini-Hochberg multiplicity correction was performed using JMP 7.0.1 (SAS Institute) and irMF 3.5 (National Institute of Statistical Sciences, NISS) software.

TABLE 1
mRNA expression measured by Affymetrix of the expression of CCR1, CCR2,
CCR5, CXCR3, CXCR4, CXCR5, CXCR6 CXCL9, CXCL10, CXCL11, CXCL12, CXCL13,
CXCL16, CCL2, CCL3, CCL4, CCL5, CCL7 and CCL13.

| | | | | | patients with |
|---|---|---|---|---|---|
| | | | | patients | Rosacea type I |
| | | Healthy | patients with | with Rosacea type I | vs Healthy |
| | | volunteers | Rosacea type I | vs Healthy volunteers | volunteers |
| GENE_SYMBOL | TITLE | Mean_Expressions | Mean_Expression | Fold_Change | Adjusted_Pvalue |
| CXCL9 | chemokine (C—X—C motif) ligand 9 | 55 | 467 | 8.5 | 1.1E−03 |
| CXCL10 | chemokine (C—X—C motif) ligand 10 | 55 | 388 | 7.1 | 2.9E−03 |
| CXCL13 | chemokine (C—X—C motif) ligand 13 | 11 | 59 | 5.1 | 1.6E−03 |
| CXCL11 | chemokine (C—X—C motif) ligand 11 | 11 | 61 | 5.7 | 1.6E−02 |
| CCL5 | chemokine (C-C motif) ligand 5 | 50 | 203 | 4.1 | 2.0E−03 |
| CXCR4 | chemokine (C—X—C motif) receptor 4 | 78 | 360 | 4.6 | 4.0E−06 |
| CCR5 | chemokine (C-C motif) receptor 5 | 67 | 175 | 2.6 | 4.0E−04 |
| CCL4 | chemokine (C-C motif) ligand 4 | 27 | 64 | 2.3 | 1.1E−02 |
| CCR1 | chemokine (C-C motif) receptor 1 | 66 | 91 | 1.4 | 3.2E−01 |
| CCR2 | chemokine (C-C motif) receptor 2 | 52 | 114 | 2.2 | 7.0E−04 |
| CXCR6 | chemokine (C—X—C motif) receptor 6 | 31 | 56 | 1.8 | 8.0E−04 |
| CCL2 | chemokine (C-C motif) ligand 2 | 248 | 423 | 1.7 | 2.1E−02 |
| CXCL16 | chemokine (C—X—C motif) ligand 16 | 230 | 323 | 1.4 | 6.3E−03 |
| CCL13 | chemokine (C-C motif) ligand 13 | 152 | 315 | 2.1 | 4.5E−02 |
| CCL7 | chemokine (C-C motif) ligand 7 | 9 | 11 | 1.3 | 6.5E−02 |
| CXCL12 | chemokine (C—X—C motif) ligand 12 | 312 | 433 | 1.4 | 1.4E−01 |
| CXCR3 | chemokine (C—X—C motif) receptor 3 | 32 | 36 | 1.1 | 3.0E−01 |
| CXCR5 | chemokine (C—X—C motif) receptor 5 | 17 | 18 | 1.1 | 2.8E−01 |

| | | | patients with | patients with |
|---|---|---|---|---|
| | | | Rosacea type II | Rosacea type II |
| | | patients with | vs Healthy | vs Healthy |
| | | Rosacea type II | volunteers | volunteers |
| GENE_SYMBOL | TITLE | Mean_Expression | Fold_Change | Adjusted_Pvalue |
| CXCL9 | chemokine (C—X—C motif) ligand 9 | 2252 | 41.1 | 3.7E−06 |
| CXCL10 | chemokine (C—X—C motif) ligand 10 | 1817 | 33.2 | 1.1E−05 |
| CXCL13 | chemokine (C—X—C motif) ligand 13 | 371 | 32.4 | 4.1E−07 |
| CXCL11 | chemokine (C—X—C motif) ligand 11 | 332 | 30.7 | 6.1E−05 |
| CCL5 | chemokine (C-C motif) ligand 5 | 425 | 8.5 | 4.1E−05 |
| CXCR4 | chemokine (C—X—C motif) receptor 4 | 630 | 8.1 | 1.1E−07 |
| CCR5 | chemokine (C-C motif) receptor 5 | 481 | 7.2 | 7.7E−08 |
| CCL4 | chemokine (C-C motif) ligand 4 | 192 | 7.0 | 4.5E−06 |
| CCR1 | chemokine (C-C motif) receptor 1 | 366 | 5.6 | 1.5E−05 |
| CCR2 | chemokine (C-C motif) receptor 2 | 220 | 4.2 | 7.5E−07 |
| CXCR6 | chemokine (C—X—C motif) receptor 6 | 122 | 3.9 | 3.2E−08 |
| CCL2 | chemokine (C-C motif) ligand 2 | 861 | 3.5 | 1.4E−05 |
| CXCL16 | chemokine (C—X—C motif) ligand 16 | 477 | 2.1 | 3.5E−06 |
| CCL13 | chemokine (C-C motif) ligand 13 | 314 | 2.1 | 5.7E−02 |
| CCL7 | chemokine (C-C motif) ligand 7 | 17 | 2.0 | 2.3E−05 |
| CXCL12 | chemokine (C—X—C motif) ligand 12 | 555 | 1.8 | 1.5E−02 |
| CXCR3 | chemokine (C—X—C motif) receptor 3 | 55 | 1.8 | 4.1E−05 |
| CXCR5 | chemokine (C—X—C motif) receptor 5 | 18 | 1.1 | 1.9E−01 |

| | | | patients with | patients with |
|---|---|---|---|---|
| | | | Rosacea type III | Rosacea type III |
| | | patients with | vs Healthy | vs Healthy |
| | | Rosacea type III | volunteers | volunteers |
| GENE_SYMBOL | TITLE | Mean_Expression | Fold_Change | Adjusted_Pvalue |
| CXCL9 | chemokine (C—X—C motif) ligand 9 | 1052 | 19.2 | 6.3E−05 |
| CXCL10 | chemokine (C—X—C motif) ligand 10 | 757 | 13.8 | 3.0E−04 |
| CXCL13 | chemokine (C—X—C motif) ligand 13 | 472 | 41.3 | 1.6E−07 |
| CXCL11 | chemokine (C—X—C motif) ligand 11 | 98 | 9.0 | 4.5E−03 |
| CCL5 | chemokine (C-C motif) ligand 5 | 296 | 5.9 | 3.0E−04 |
| CXCR4 | chemokine (C—X—C motif) receptor 4 | 470 | 6.0 | 8.8E−07 |
| CCR5 | chemokine (C-C motif) receptor 5 | 303 | 4.5 | 3.0E−06 |
| CCL4 | chemokine (C-C motif) ligand 4 | 109 | 4.0 | 3.0E−04 |
| CCR1 | chemokine (C-C motif) receptor 1 | 159 | 2.4 | 9.6E−03 |
| CCR2 | chemokine (C-C motif) receptor 2 | 170 | 3.3 | 1.0E−05 |
| CXCR6 | chemokine (C—X—C motif) receptor 6 | 82 | 2.6 | 3.6E−06 |
| CCL2 | chemokine (C-C motif) ligand 2 | 625 | 2.5 | 4.0E−04 |
| CXCL16 | chemokine (C—X—C motif) ligand 16 | 371 | 1.6 | 5.0E−04 |
| CCL13 | chemokine (C-C motif) ligand 13 | 242 | 1.6 | 2.3E−01 |
| CCL7 | chemokine (C-C motif) ligand 7 | 11 | 1.3 | 1.0E−01 |
| CXCL12 | chemokine (C—X—C motif) ligand 12 | 596 | 1.9 | 6.7E−03 |
| CXCR3 | chemokine (C—X—C motif) receptor 3 | 46 | 1.4 | 3.0E−03 |
| CXCR5 | chemokine (C—X—C motif) receptor 5 | 21 | 1.2 | 8.6E−03 |

The table 1 shows the mRNA of CXCR3, slightly induced in rosacea subtype II and its specific ligands including CXCL9, CXCL10 and CXCL11, strongly induced in all rosacea subtypes indicating a strong chemoattraction for T cells (preferentially Th1 cells). The expression of CXCR4 is induced in three rosacea subtype but the mRNA of its ligand, CXCL12, is not modulated in rosacea, but its expression in human skin is clearly demonstrated. Despite the non modulation of CXCR5, the expression of its ligand CXCL13 is significantly up-regulated in rosacea lesions, promoting B cell migration. Expression of CXCR6 and its unique ligand CXCL16 are induced mainly in rosacea subtype II. The mRNA of CCR2 and chemokines binding to this receptor, CCL2, CCL7, CCL13, are found to be increased. The others receptors involved in leukocyte recruitment such as CCR5 and CCR1 and their specific ligands CCL3, CCL4, CCL5, CCL7 are also induced in rosacea lesions.

### Cytokine Extraction and Assay:

Proteins were extracted from skin biopsies in healthy volunteers and from lesional skin in patients with rosacea (subtype I or II). Cytokines were dosed in the protein extracts using Luminex assays (Millipore & Procarta cytokine dosage kits). The cytokine quantities were normalized to the total concentration of protein. Paired P-values were calculated for each cytokine.

TABLE 2
Expression of cytokines measured by Luminex: expression of CXCL10, CXCL11,
CXCL12, CXCL13, CCL2, CCL4, CCL5, CCL7.

| | | | | | | Rosacea | | Rosacea | |
|---|---|---|---|---|---|---|---|---|---|
| | | | Rosacea | Rosacea | Healphy | Subtype 1/ | | Subtype 2/ | |
| Protein | | | Subtype 1 | Subtype 2 | skin | Healthy | | Healthy | |
| | | Primary | Conc. | Conc. | Conc. | Fold | p- | Fold | p- |
| Symbol | Name | Access. No | [pg/mg] | [pg/mg] | [pg/mg] | modulation | value | modulation | value |
| IP-10 | C—X—C motif | P02778 | 86 | 25 | 6 | 16 | <0.05 | 4.5 | NS |
| | chemokine | | | | | | | | |
| | 10 | | | | | | | | |
| SDF-1/ | C—X—C motif | P48061 | 10 | 13 | 2 | 3.9 | <0.01 | 5.2 | <0.05 |
| CXCL12 | chemokine | | | | | | | | |
| | 12 | | | | | | | | |
| MCP-1 | C—X—C motif | P13500 | 55 | 48 | 20 | 2.8 | <0.01 | 2.5 | <0.05 |
| | chemokine 2 | | | | | | | | |
| BLC/ | C—X—C motif | O43927 | 1.5 | 1.9 | 0.6 | 2.8 | <0.05 | 3.5 | <0.01 |
| CXCL13/ | chemokine | | | | | | | | |
| BCA-1 | 13 | | | | | | | | |
| MIP-1 | C—X—C motif | P13236 | 2.3 | 2.0 | 0.8 | 2.8 | <0.05 | 2.5 | <0.05 |
| beta | chemokine 4 | | | | | | | | |
| RANTES | C—X—C motif | P13501 | 352 | 231 | 181 | 1.9 | NS | 1.3 | NS |
| | chemokine 5 | | | | | | | | |
| I-TAC/ | C—X—C motif | O14625 | 18 | 18 | 10 | 1.8 | NS | 1.9 | NS |
| CXCL11 | chemokine | | | | | | | | |
| | 11 | | | | | | | | |
| MCP-3 | C—X—C motif | P80098 | 1.0 | 1.0 | 0.8 | 1.2 | NS | 1.2 | NS |
| | chemokine 7 | | | | | | | | |

NS: not significant

Table 2 shows a significant up-regulation of the protein expression level of CXCL10, CXCL12, CCL2, CXCL13 and CCL4 in rosacea lesional skin (type I and II) in comparison to healthy skin, indicating a leukocyte recruitment.

1. A method for diagnosing rosacea, the method comprising detecting the presence of the DNA or the mRNA encoding CCR1, CCR2, CCR5, CXCR3, CXCR4, CXCR5, CXCR6, CXCL9, CXCLIO, CXCL11, CXCL12, CXCL13, CXCL16, CCL2, CCL3, CCL4, CCL5, CCL7, and CCL13 and also the corresponding proteins in an individual subject in need thereof, as markers for rosacea and correlating the presence of the markers with a diagnosis of rosacea.

2. A method for diagnosing rosacea, the method comprising the following steps:
a) detecting a level of expression of at least one marker selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13, in a sample taken from an individual;
b) detecting a level of expression of at least one marker selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 in a sample taken from a healthy individual;
c) comparing a difference in level of expression of at least one marker selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 in the individual with the healthy individual; and
d) identitying an overexpression of at least one of the markers selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 as an indicator of rosacea, and thus diagnosing rosacea based on the existence of the indicator.

3. A method for monitoring progression or variation of rosacea, the method comprising the following steps:
a) taking a biological sample from the individual;
b) analyzing a level of expression of at least one of markers selected from CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 in a sample taken and in which identifying a variation in the expression level of at least one of the markers as an indicator of the progression of rosacea.

4. A method for monitoring efficacy of a treatment intended for treating rosacea, the method comprising the following steps:
a) administering the treatment to an individual identified as having one or more of the symptoms of rosacea;
b) taking a biological sample from the individual;
c) analyzing a level of expression of at least one marker selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 and identify an overexpression as an indicator of the efficacy of the treatment of rosacea.

5. An in vitro screening method of drug candidates, the method comprising determining capacity of the candidate to modulate transactivation activity of a marker selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 and identifying those candidates that modulated the transactivation activity as candidates for treating rosacea.

6. An in vitro screening method of modulators for identifying drug candidates, the method comprising the following steps:
a) collecting at least two biological samples: one mimicking a rosacea lesion, and one mimicking a healthy condition;
b) contacting at least one sample or a mixture of samples with one or more drug candidates to be tested;
c) detecting an expression or biological function of at least one marker selected from the group consisting of CCR1, CCR2, CCR5, CXCR3, CXCR4, CXCR5, CXCR6, CXCL9, CXCL10, CXCL11, CXCL12, CXCL13, CXCL16, CCL2, CCL3, CCL4, CCL5, CCL7, and CCL13 in the biological samples or mixture obtained in b); and
d) selecting drug candidates that downregulate the expression or biological function of CCR1 CCR2, CCR5, CXCR3, CXCR4, CXCR5, CXCR6, CXCL9, CXCL10, CXCL11, CXCL12, CXCL13, CXCL16, CCL2, CCL3, CCU-, CCL5, CCL7, or CCL13 measured in said samples or mixtures obtained in b) and comparing the levels with levels obtained from a sample not mixed with the drug candidate.

7. A method for treating rosacea and/or rosacea associated disorders, the method comprising administering modulators identified by the screening method as defined in claim 5 to an individual subject in need thereof.

8. A method for treating rosacea, the method comprising administering a modulator of at least one marker of leukocyte trafficking selected from the group of modulators consisting of:
2-thiophen-2-yl-5-[-5-[5-(5-thiophen-2-ylthiophen-2-yl)thiophen-2-yl]thiophen-2-yl]thiophene; 1,4-cis-1-(1-Cycloocten-1-ylmethyl)-4-[[(2,7-dichloro-9H-xanthen-9-yl)carbonyl]amino]-1-ethylpiperidinium iodide, (7R,7aS)-2-Chloro-4-(7-hydroxy-1,3-dioxotetrahydropyrrolo[1,2-c]imidazol-2-yl)-3-methylbenzonitrile, [5-chloro-2-[2-[(2R)-4-[(4-fluorophenyl)methyl]-2-methylpiperazin-1-yl]-2-oxoethoxy]phenyl]urea hydrochloride, compounds disclosed in patents WO 2010/036632, WO 2009/134666 and WO 2009/137338, WO 98/56771, U.S. Pat. No. 6,812,230, US 2008/0139602, WO/2003/105853, WO 2009/082526, WO/1998/038167, WO 2008/011392, WO/2011/056440, WO/2011/049917, WO/2006/133802, WO2008/103126 and WO2009/011653; (5E)-8-[4-(2-butoxyethoxy)phenyl]-1-(2-methylpropyl)-N-[4-[(3-propylimidazol-4-yl) methylsulfinyl]phenyl]-3,4-dihydro-2H-1-benzazocine-5-carboxamide; methanesulfonic acid, 6-Methyl-1′-[2-(5-methyl-2-phenyl-4-oxazolyl)ethyl]-spiro[4H-3,1-benzoxazine-4,4′-piperidin]-2(1H)-one, 2-[(Isopropylaminocarbonyl)amino]-N-[2-[[cis-2-[[4-(methylthio)benzoyl]amino]cyclohexyl]amino]-2-oxoethyl]-5-(trifluoromethyl)benzamide, 1′-[2-[4-(Trifluoromethyl)phenyl]ethyl]-spiro[4H-3,1-benzoxazine-4,4′-piperidin]-2(1H-one hydrochloride, compounds disclosed in patents WO2009/076404, WO2006/012135, WO2004/069810, WO2006/036527, WO2008/109238, WO2008/145681, WO2007/130712, WO2007/106797, WO2005/118574, WO2006/015986, WO2011/073155, WO2011/073154, WO2011/042399, WO2010/070032, WO2007/053495, WO2007/053498, WO2007/053499, WO/2010/068663, WO2010/121011, WO2010/121046, WO2009/003861, WO2010/121036, WO2006/076644, WO2008/008375, WO2008/010934, WO2010/074409; 4,4-difluoro-N-[(1S)-3-[(1R,5S)-3-(3-methyl-5-propan-2-yl-1,2,4-triazol-4-yl)-8-azabicyclo[3.2.1]octan-8-yl]-1-phenylpropyl]cyclohexane-1-carboxamide, (5E)-8-[4-(2-butoxyethoxy)phenyl]-1-(2-methylpropyl)-N-[4-[(S)-(3-propylimidazol-4-)methylsulfinyl]phenyl]-3,4-dihydro-2H-1-benzazocine-5-carboxamide, (4-nitrophenyl)methyl N-[1-[[(3S,4R)-1-(cyclopentanecarbonyl)-4-hydroxy-4-phenylpyrrolidin-3-yl]methyl]piperidin-4-yl}-N-prop-2-enylcarbamate, 1-acetyl-N-[3-[4-[(4-carbamoylphenyl)methyl]piperidin-1-yl]propyl]-N-(3-chloro-4-methylphenyl)piperidine-4-carboxamide, (4,6-dimethylpyrimidin-5-yl)-[4-[(3S)-4-[(1R)-2-methoxy-1-[4-(trifluoromethyl)phenyl]ethyl]-3-methylpiperazin-1-yl]-4-methylpiperidin-1-yl]methanone, 4-[4-[[(9R)-11-butyl-9-[(R)-cyclohexyl(hydroxy)methyl]-7,10-dioxo-3,8,11-triazaspiro[5.5]undecan-3-yl]methyl]phenoxy]benzoic acid; N-[(1R)-1-[3-(4-ethoxyphenyl)-4-oxopyrido[2,3-d]pyrimidin-2-yl]methyl]-N-(pyridin-3-ylmethyl)-2-[4-(trifluoromethoxy)phenyl]acetamide, compounds disclosed in patents: WO2007/062175, WO2002/083143, WO2009/094168, WO2002/085861, WO2004/075863, WO2006/088836, WO2008/079279, WO2011/084985, WO2007/090826, WO2007/090836, WO2008/008453, WO2007/109238, WO2007/047202, WO2006/088921 , WO2006/088840, WO2006/088920, WO2006/088919, WO2006/091428, WO2007/002742, WO2007/064553; 1-[[4-(1,4,8,11-tetrazacyclotetradec-1-ylmethyl)phenyl]methyl]-1,4,8,11-tetrazacyclotetradecane, N-(pyridin-2-ylmethyl)-1-[3-(1,4,8,11-tetrazacyclotetradec-1-ylmethyl)phenyl]methanamine, compounds disclosed in patents: WO2004/087068, WO2006/074428, WO2008/008852, WO2006/126188, WO2006/074426, WO2008/150689, WO2010/025416, WO2009/004054, WO2007/074871, WO2008/008854, WO2006/074426; and compounds disclosed in patents: WO2010/053547, WO2008/151211.
